(19) United States
(12) Patent Application Publication
(10) Pub. No.: US 2010/0324311 A1
(43) Pub. Date: Dec. 23, 2010

(54) METHOD FOR THE PRODUCTION OF CRUDE TRIOXANE

(75) Inventors: Markus Siegert, Heidelberg (DE); Neven Lang, Mannheim (DE); Joachim Thiel, Neustadt (DE); Eckhard Stroefer, Mannheim (DE); Christoph Sigwart, Weinheim (DE)

Correspondence Address: CONNOLLY BOVE LODGE & HUTZ, LLP, P O BOX 2207, WILMINGTON, DE, 19899, US

(73) Assignee: BASF SE, Ludwigshafen (DE)

(21) Appl. No.: 12/809,338

(22) PCT Filed: Dec. 11, 2008
(86) PCT No.: PCT/EP2008/067287

§ 371 Date: Jun. 18, 2010

(30) Foreign Application Priority Data
Dec. 19, 2007 (EP) 07150114.2

(51) Int. Cl. C07D 323/06

(52) U.S. Cl. 549/368

(57) ABSTRACT

A process for preparing crude trioxane (1) having a concentration in the range from 50 to 75% by weight of trioxane by trimerization of formaldehyde from an aqueous formaldehyde solution (2) in the presence of an acid catalyst (3) and concentration of the trioxane from the reaction mixture from the trimerization by distillation, wherein
the trimerization of the formaldehyde and the concentration of the trioxane from the reaction mixture from the trimerization are carried out in a single column (K) which
is divided by a horizontal dividing plate (T) into a lower column region (A) and an upper column region (B),
with the lower column region (A) and the upper column region (B) being connected by an external vapor line and an external liquid line, and
a reactive distillation in which the formaldehyde is trimerized to trioxane and the trioxane in the reaction mixture is concentrated to the solubility limit of formaldehyde is carried out in the lower column region (A) at a pressure in the range from 1 to 5 bar and
a distillation in which the trioxane is concentrated to give crude trioxane having a concentration in the range from 50 to 75% by weight is carried out in the upper column region (B) at a pressure in the range from 200 to 900 mbar, is proposed.

The invention relates to a process for preparing crude trioxane having a concentration in the range from 50 to 75% by weight by trimerization of formaldehyde from an aqueous formaldehyde solution in the presence of an acid catalyst and concentration of the trioxane from the reaction mixture from the trimerization by distillation.

Trioxane is obtained predominantly by trimerization of formaldehyde from aqueous formaldehyde solutions by homogeneous or heterogeneous catalysis in the presence of acid catalysts. A problem is that formaldehyde tends to precipitate as solid with formation of paraformaldehyde, with the temperature at which the solid begins to precipitate decreasing with increasing formaldehyde content in an aqueous solution. As an approximation, at least for formaldehyde concentrations in the range from 30 to 70% by weight, aqueous formaldehyde solutions have to be heated to a temperature which is about 10° C. higher than the concentration of formaldehyde in % by weight in order to avoid precipitation of a solid. Thus, a 50% strength aqueous formaldehyde solution has to be heated to about 60-70° C. and a 60% strength formaldehyde solution has to be heated to about 70-80° C. in order to keep the formaldehyde in solution.

The trioxane obtained by trimerization of formaldehyde is, if appropriate together with further monomers, predominantly used as monomer for preparing polyoxymethylene homopolymers or copolymers.

Polymerization-grade trioxane has to meet particular specifications and will hereinafter be referred to as pure trioxane. This is a stream having a minimum content of 97.5% by weight of trioxane or even 99% by weight of trioxane or 99.5% by weight of trioxane. A stream having a minimum content of 99.9% by weight of trioxane can be referred to as high-purity trioxane.

The production of pure trioxane by concentration of the reaction mixture from the trimerization of formaldehyde is made difficult in process engineering terms by trioxane, formaldehyde and water forming a ternary azeotrope which at a pressure of 1 bar has the composition 69.5% by weight of trioxane, 5.4% by weight of formaldehyde and 25.1% by weight of water and whose composition is strongly pressure dependent, with the proportion of trioxane being higher at lower pressures.

The concentration of the trioxane/formaldehyde/water mixture to give pure trioxane is therefore carried out industrially in such a way that the azeotrope problems are avoided, for example by extraction as described in DE-A 1668867 or by pervaporation as described in DE-A 197 32 291. However, these separation processes are expensive due to the costs for extractants or a pervaporation unit and the handling therefor.

DE 103 61 516 has therefore proposed a separation by pressure swing rectification in which the trioxane/formaldehyde/water mixture from the trimerization of formaldehyde is fractionally distilled at different pressures in a plurality of columns. Here, formaldehyde is separated off at low pressure in a first column, i.e. an essentially trioxane-free formaldehyde/water stream and a trioxane-comprising aqueous stream having a low formaldehyde content. This trioxane-comprising aqueous stream having a low formaldehyde content is separated in a second column which is operated at a higher pressure than the first column into pure trioxane and a trioxane/formaldehyde/water mixture having a lower trioxane content from which essentially pure water is separated off in a further column to give a trioxane/formaldehyde/water mixture which is recycled to the first distillation column.

However, this process incurs high capital and operating costs for the plurality of distillation columns.

It was therefore an object of the invention to provide a technically simple process by means of which crude trioxane having a very high trioxane concentration is obtained by trimerization of formaldehyde in such a way that the further concentration to give polymerization-grade pure trioxane is less complicated.

This object is achieved by a process for preparing crude trioxane having a concentration in the range from 50 to 75% by weight of trioxane by trimerization of formaldehyde from an aqueous formaldehyde solution in the presence of an acid catalyst and concentration of the trioxane from the reaction mixture from the trimerization by distillation, wherein
the trimerization of the formaldehyde and the concentration of the trioxane from the reaction mixture from the trimerization are carried out in a single column which
is divided by a horizontal dividing plate into a lower column region A and an upper column region B,
with the lower column region A and the upper column region B being connected by an external vapor line and an external liquid line, and
a reactive distillation in which the formaldehyde is trimerized to trioxane and the trioxane in the reaction mixture is concentrated to the solubility limit of formaldehyde is carried out in the lower column region A at a pressure in the range from 1 to 5 bar and
a distillation in which the trioxane is concentrated to give crude trioxane having a concentration in the range from 50 to 75% by weight is carried out in the upper column region B at a pressure in the range from 200 to 900 mbar.

It has been found that it is possible to carry out the synthesis and concentration of trioxane in a single column which is, as a result of appropriate choice of the operating parameters temperature and pressure, matched to the solubility behavior of formaldehyde and trioxane in aqueous mixtures, operated in such a way that solid does not precipitate at any point in the column. The plant availability is accordingly increased.

As a result of a process in which synthesis and concentration of trioxane are carried out in a single apparatus being made available according to the invention, the capital costs are lower compared to known processes.

The trioxane synthesis generally starts out from an aqueous formaldehyde solution comprising from 45 to 75% by weight of formaldehyde and water as balance. This solution can, if necessary, be obtained from an aqueous formaldehyde solution having a lower formaldehyde concentration in a preceding concentration step. The concentration step can be carried out, for example, in an evaporator, preferably a falling film evaporator, as described, for example, in DE-A 199 25 870.

The trimerization of formaldehyde is carried out in the presence of acid catalysts present in homogeneous or heterogeneous form, e.g. ion exchangers, resins, zeolites, sulfuric acid or paratoluenesulfonic acid.

The synthesis of trioxane by trimerization of formaldehyde is, according to the invention, carried out in the lower column region A of a column K at a pressure in the range from 1 to 5 bar and a temperature corresponding to this pressure in the range from about 100 to 200° C.

Trimerization of the formaldehyde forms a trioxane-comprising reaction mixture which is concentrated in trioxane by rectification in the lower column region on separation-active internals. The number of theoretical plates in the lower column region is selected in the range from about 1 to 20 so that the reaction mixture at the upper end of the lower column region is concentrated as much as possible in trioxane while ensuring that the vapor stream taken off from the upper end of the lower column region does not lead to precipitation of solids after being introduced via an external line into the lower region of the upper column region which is operated at a lower pressure than the lower column region.

The column is divided in a liquid-tight and gastight manner into a lower column region and an upper column region by means of a horizontal dividing plate. The upper end of the lower column region and the lower end of the upper column region are connected to one another in each case via an external line located outside the column.

In the upper column region, further concentration of trioxane takes place at a subatmospheric pressure in the range from 200 to 900 mbar. The concentration by distillation is more preferably carried out at a pressure in the range from 500 to 700 mbar.

The number of theoretical plates in the distillation section of the column, i.e. in the from upper column region, is selected so that highly concentrated crude trioxane can be taken off at the top of the column while ensuring that solid does not precipitate at any place in the column. The number of theoretical plates in the upper column region can preferably be from 1 to 25.

A water-comprising stream can advantageously be taken off in liquid or gaseous form from the upper or lower column region and be recycled to a subsequent process stage.

A crude trioxane stream comprising from 50 to 75% by weight of trioxane, or from 60 to 75% by weight of trioxane, is taken off at the top of the column.

The invention is illustrated below with the aid of an example and a drawing.

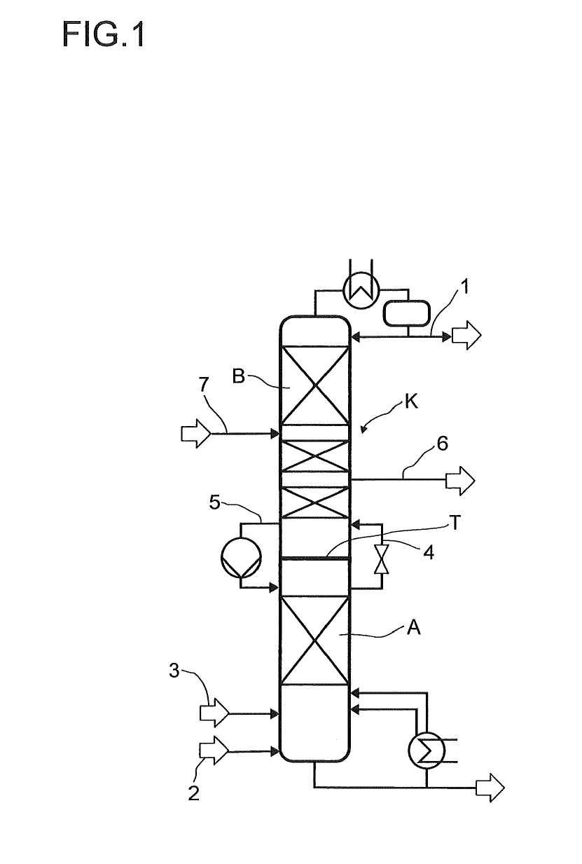

The single FIGURE, FIG. 1, shows a column K having a horizontal dividing wall T which divides the column into a lower column region A and an upper column region B.

Crude trioxane, stream 1, is taken off at the top of the column. In the lower part of the lower column region A, an aqueous formaldehyde solution, stream 2, and acid catalyst stream 3 are fed in. The lower column region A is connected to the upper column region B via an external vapor line and an external liquid line 5.

In the preferred embodiment depicted in the figure, a water-comprising stream 6 is taken off in liquid form from the upper column region B.

A trioxane- and/or formaldehyde-comprising stream 7 can preferably be fed, as shown in the FIGURE, into the upper column region B.

1-5. (canceled)

6. A process for preparing crude trioxane having a concentration in the range from 50 to 75% by weight of trioxane by trimerization of formaldehyde from an aqueous formaldehyde solution in the presence of an acid catalyst and concentration of the trioxane from the reaction mixture from the trimerization by distillation, wherein
the trimerization of the formaldehyde and the concentration of the trioxane from the reaction mixture from the trimerization are carried out in a single column which
is divided by a horizontal dividing plate into a lower column region and an upper column region,
with the lower column region and the upper column region being connected by an external vapor line and an external liquid line, and
a reactive distillation in which the formaldehyde is trimerized to trioxane and the trioxane in the reaction mixture is concentrated to the solubility limit of formaldehyde is carried out in the lower column region at a pressure in the range from 1 to 5 bar and
a distillation in which the trioxane is concentrated to give crude trioxane having a concentration in the range from 50 to 75% by weight is carried out in the upper column region at a pressure in the range from 200 to 900 mbar.

7. The process of claim 6, wherein crude trioxane having a concentration in the range from 60 to 75% by weight is prepared.

8. The process of claim 6, wherein the reactive distillation is carried out in the lower column region at a pressure in the range from 1.25 to 1.75 bar and the distillation in the upper column region is carried out at a pressure in the range from 500 to 700 mbar.

9. The process of claim 6, wherein from 1 to 20 theoretical plates are provided in the lower column region and from 1 to 25 theoretical plates are provided in the upper column region.

10. The process of claim 6, wherein a water-comprising, liquid or gaseous stream is taken off from the upper column region or the lower column region and is recycled to a downstream process stage.
